(19) United States
(12) Patent Application Publication
(10) Pub. No.: US 2026/0283734 A1
(43) Pub. Date: Sep. 24, 2026

(54) ANTI-INVERSION BUMPER FOR EXPANDABLE ABLATION CATHETER ASSEMBLY

(71) Applicant: Boston Scientific Scimed, Inc., Maple Grove, MN (US)

(72) Inventors: Benjamin Michael WILKE, Shoreview, MN (US); James HOLMBERG, Champlin, MN (US)

(21) Appl. No.: 19/572,685

(22) Filed: Mar. 19, 2026

Related U.S. Application Data
(60) Provisional application No. 63/775,802, filed on Mar. 21, 2025.

(51) Int. Cl. A61B 90/00; A61B 18/14; A61L 29/06; A61B 18/00; A61M 25/00

(52) U.S. Cl. CPC A61B 90/03; A61B 18/1492; A61L 29/06; A61B 2018/00267; A61B 2018/00577; A61B 2090/034; A61M 25/0074

(57) ABSTRACT

A medical device includes an outer member having a proximal end and an opposite distal end, and an inner member movable within the outer member. An expandable assembly extends distally from the distal end of the outer member. The expandable assembly includes a distally located central hub portion affixed to the inner member and a plurality of splines. Each of the plurality of splines includes a distal end portion extending from the central hub portion, and a proximal end portion attached to the distal end of the outer member. At least one electrode is located on each of the plurality of splines. A bumper is positioned on the inner member within the expandable assembly. The bumper is configured to abut an inner surface of the distal end portion of the plurality of splines to prevent inversion of the plurality of splines.

### CROSS REFERENCE TO RELATED APPLICATIONS

The present application claims priority to U.S. Provisional Patent Application No. 63/775,802, filed Mar. 21, 2025, the disclosure of which is incorporated herein in its entirety.

### TECHNICAL FIELD

The present disclosure relates to medical systems and methods for ablating tissue in a patient. More specifically, the present disclosure relates to medical systems and methods for ablation of tissue by electroporation.

### BACKGROUND

Ablation procedures are used to treat many different conditions in patients. Ablation can be used to treat cardiac arrhythmias, benign tumors, cancerous tumors, and to control bleeding during surgery. Usually, ablation is accomplished through thermal ablation techniques including radio-frequency (RF) ablation and cryoablation. In RF ablation, a probe is inserted into the patient and radio frequency waves are transmitted through electrodes located on the probe to the surrounding tissue. The radio frequency waves generate heat, which destroys surrounding tissue and cauterizes blood vessels.

Some probes include expanding assemblies, or cages, having splines that carry one or more electrode. During use, the splines sometimes prolapse or invert across the centerline of the expandable assembly, or cage. This can occur if the device is delivered through a sheath or bent significantly when traveling through narrow passageways. This prolapse or inversion can result in inoperability of the device. The improvements herein are directed to preventing prolapse or inversion of splines forming an expandable assembly.

### SUMMARY

Example 1 is a medical device including an outer member having a proximal end and an opposite distal end, and an inner member movable within the outer member. An expandable assembly extends distally from the distal end of the outer member. The expandable assembly includes a distally located central hub portion affixed to the inner member and a plurality of splines. Each of the plurality of splines includes a distal end portion extending from the central hub portion, and a proximal end portion attached to the distal end of the outer member. A bumper is positioned on the inner member within the expandable assembly. The bumper is configured to abut an inner surface of the distal end portion of the plurality of splines to prevent inversion of the plurality of splines.

Example 2 is the medical device of Example 1, further comprising at least one electrode located on each of the plurality of splines.

Example 3 is the medical device of Example 2, wherein the at least one electrode is an ablation electrode, sensing electrode, mapping electrode, pacing electrode, or cauterizing electrode.

Example 4 is the medical device of any of Examples 1 to 3, wherein the bumper includes a bumper proximal end, an opposite bumper distal end, and an outer profile extending from the bumper distal end to the bumper proximal end.

Example 5 is the medical device of Example 4, wherein the outer profile tapers from a smaller diameter at the bumper distal end to a larger diameter at the bumper proximal end.

Example 6 is the medical device of any of Examples 1 to 5, wherein the bumper includes a plurality of fins, the plurality of fins being spaced apart along the outer profile.

Example 7 is the medical device of Example 6, wherein the plurality of fins are configured to be received between adjacent ones of the plurality of splines.

Example 8 is the medical device of any of Examples 1 to 7, wherein the bumper is a polymeric material.

Example 9 is the medical device of Example 8, wherein the polymeric material is silicone or PEBAX.

Example 10 is the medical device of any of Examples 1 to 9, wherein the bumper is cylindrical or conical.

Example 11 is the medical device of any of Examples 1 to 10, wherein the bumper includes a lumen configured to receive the inner member.

Example 12 is the medical device of any of Examples 1 to 11, wherein the bumper is formed of a compliant material.

Example 13 is the medical device of any of Examples 1 to 12, wherein the bumper includes a first portion having a first diameter, and a second portion having a second diameter that is larger than the first diameter.

Example 14 is the medical device of Example 13, wherein the first portion is distal to the second portion.

Example 15 is the medical device of any of Examples 1 to 5, wherein the inner member has a portion of increased diameter forming the bumper.

Example 16 is a medical device having an outer member having a proximal end and an opposite distal end, and an inner member movable within the outer member. An expandable assembly extends distally from the distal end of the outer member. The expandable assembly includes a distally located central hub portion affixed to the inner member and a plurality of splines. Each of the plurality of splines includes a distal end portion extending from the central hub portion, and a proximal end portion attached to the distal end of the outer member. At least one electrode is located on each of the plurality of splines. A bumper is positioned on the inner member within the expandable assembly. The bumper is configured to abut an inner surface of the distal end portion of the plurality of splines to prevent inversion of the plurality of splines.

Example 17 is the medical device of Example 16, wherein the at least one electrode is an ablation electrode, sensing electrode, mapping electrode, pacing electrode, or cauterizing electrode.

Example 18 is the medical device of Example 16, wherein the bumper includes a bumper proximal end, an opposite bumper distal end, and an outer profile extending from the bumper distal end to the bumper proximal end.

Example 19 is the medical device of Example 18, wherein the outer profile tapers from a smaller diameter at the bumper distal end to a larger diameter at the bumper proximal end.

Example 20 is the medical device of Example 16, wherein the bumper includes a plurality of fins, the plurality of fins being spaced apart along the outer profile.

Example 21 is the medical device of Example 20, wherein the plurality of fins are configured to be received between adjacent ones of the plurality of splines.

Example 22 is the medical device of Example 16, wherein the bumper is a polymeric material.

Example 23 is the medical device of Example 22, wherein the polymeric material is silicone or PEBAX.

Example 24 is the medical device of Example 16, wherein the bumper is cylindrical or conical.

Example 25 is the medical device of Example 16, wherein the bumper includes a lumen configured to receive the inner member.

Example 26 is the medical device of Example 16, wherein the bumper is formed of a compliant material.

Example 27 is the medical device of Example 16, wherein the bumper includes a first portion having a first diameter, and a second portion having a second diameter that is larger than the first diameter.

Example 28 is the medical device of Example 27, wherein the first portion is distal to the second portion.

Example 29 is the medical device of Example 16, wherein the inner member has a portion of increased diameter forming the bumper.

Example 30 is a medical device including an outer member having a proximal end and an opposite distal end, and an inner member movable within the outer member. An expandable assembly extends distally from the distal end of the outer member. The expandable assembly includes a distally located central hub portion affixed to the inner member and a plurality of splines. Each of the plurality of splines includes a distal end portion extending from the central hub portion, and a proximal end portion attached to the distal end of the outer member. A cylindrical or conical bumper is positioned on the inner member within the expandable assembly. The bumper is configured to abut an inner surface of the distal end portion of the plurality of splines to prevent inversion of the plurality of splines.

Example 31 is the medical device of Example 30, further comprising at least one electrode located on each of the plurality of splines.

Example 32 is the medical device of Example 31, wherein the at least one electrode is an ablation electrode, sensing electrode, mapping electrode, pacing electrode, or cauterizing electrode.

Example 33 is a medical device including an outer member having a proximal end and an opposite distal end, and an inner member movable within the outer member. An expandable assembly extends distally from the distal end of the outer member. The expandable assembly includes a distally located central hub portion affixed to the inner member and a plurality of splines. Each of the plurality of splines includes a distal end portion extending from the central hub portion and a proximal end portion attached to the distal end of the outer member. A bumper is positioned on the inner member within the expandable assembly. The bumper is configured to abut an inner surface of the distal end portion of the plurality of splines to prevent inversion of the plurality of splines. The bumper includes a first portion having a first diameter, and a second portion having a second diameter that is larger than the first diameter.

Example 34 is the medical device of Example 33, wherein the first portion is proximal of the second portion.

Example 35 is the medical device of Example 33, wherein the first portion is distal of the second portion.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a perspective view of a handle for use with a medical device, in accordance with embodiments of the disclosure.

FIG. 2A is a perspective view of a distal portion of an ablation catheter with an expandable assembly in a stored configuration, in accordance with embodiments of the disclosure.

FIGS. 2B and 2C illustrate perspective views of a distal portion of an ablation catheter with an expandable assembly in various expanded configurations, in accordance with embodiments of the disclosure.

FIG. 3 is a perspective view of a distal portion of an ablation catheter having a bumper positioned on the inner member within the expandable assembly to prevent spline inversion, in accordance with embodiments of the disclosure.

FIGS. 4A-4D illustrate perspective views of various bumpers, in accordance with embodiments of the disclosure.

FIG. 5 is a perspective view of a distal portion of an ablation catheter having a bumper with a plurality of fins, in accordance with embodiments of the disclosure.

FIG. 6A is a front view of the bumper of FIG. 5, in accordance with embodiments of the disclosure.

FIG. 6B is a side view of the bumper of FIG. 5, in accordance with embodiments of the disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

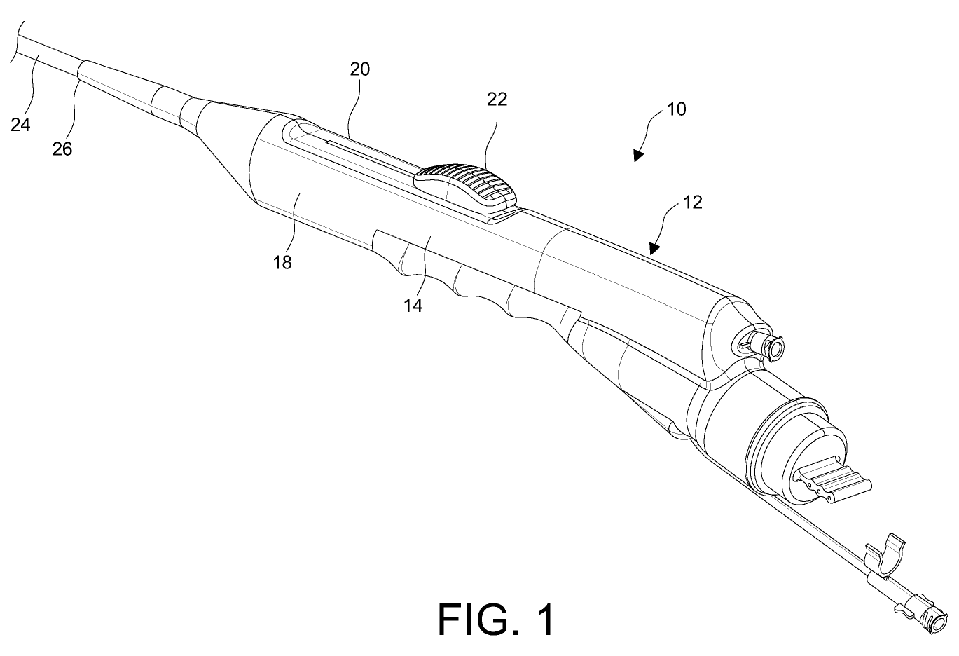

FIG. 1 is a perspective view of a handle 10 for use with a medical device, in accordance with embodiments of the disclosure. The handle 10 includes a housing 12 having an outer surface 14 and an inner cavity (not shown). The housing is formed of a left housing portion 18 and a right housing portion 20 joined together to create the inner cavity. An actuator 22 is moveable relative to the housing 12 and is configured to translate a portion of an elongated medical device. The elongated medical device is configured to change shape or arrangement by manipulation of the actuator 22. The actuator 22 is free to slide proximally or distally relative to the handle 10 to change a shape of an expandable assembly 34 located at a distal portion of the medical device.

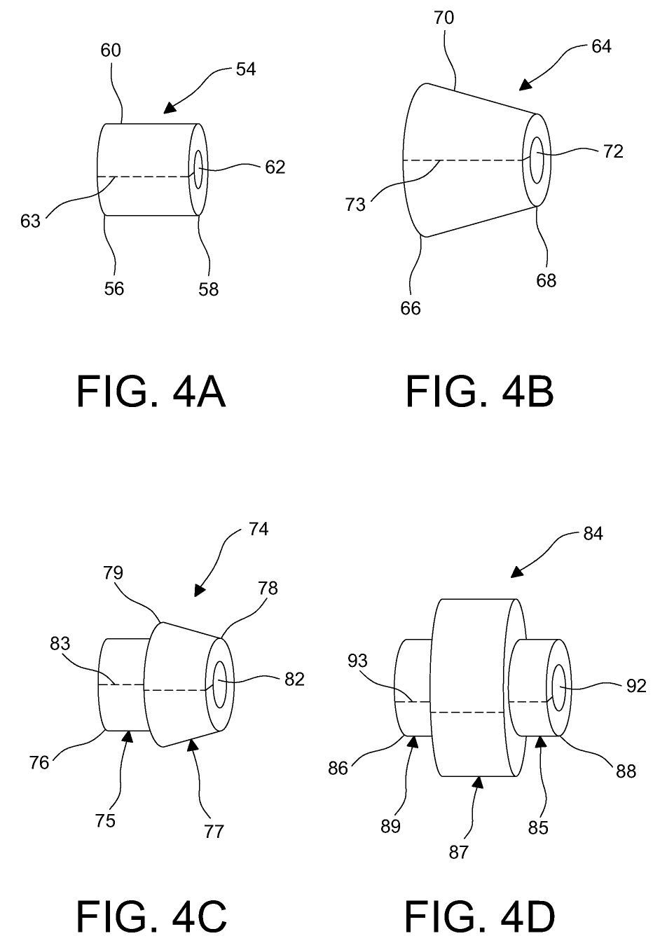

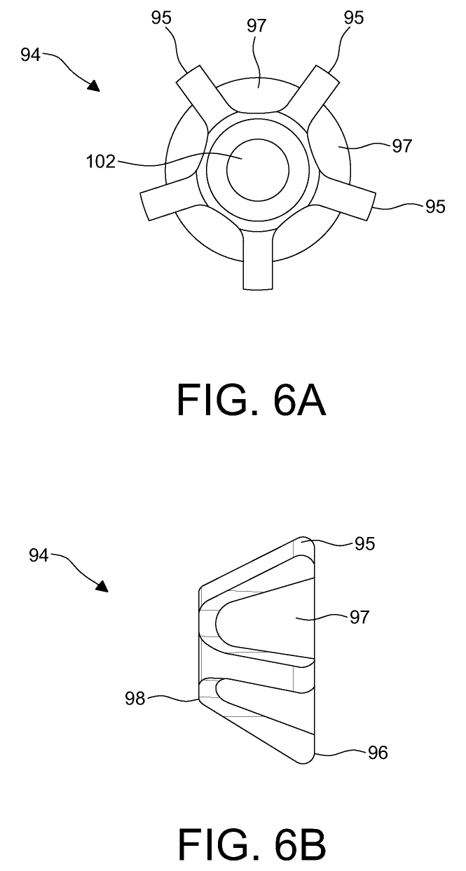

In some embodiments, the elongated medical device includes an outer member 24 connected to a distal end 26 of the handle 10, and an inner member 28 (FIGS. 4 and 6) longitudinally moveable within the outer member 24. Movement of the inner member 28 relative to the outer member 24 causes the expandable assembly 34 to move between a stored and an expanded configuration.

The actuator 22 is configured to be moved by a user’s thumb. As the actuator 22 is moved longitudinally with respect to the housing 12, the inner member 28 moves longitudinally within the outer member 24 to adjust the arrangement of the expandable assembly 34.

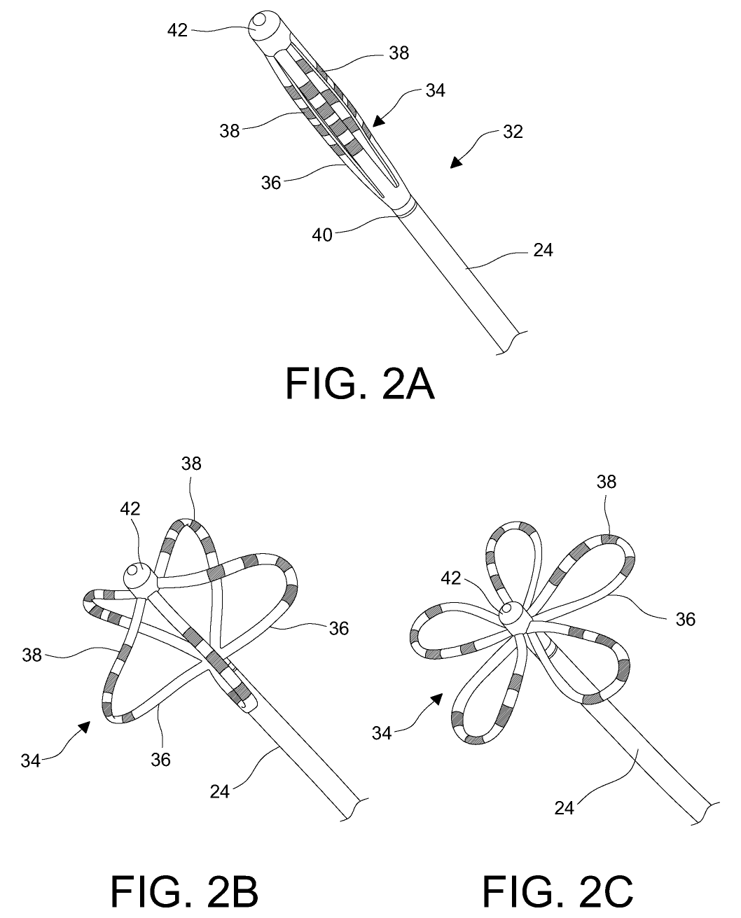

FIG. 2A illustrates an elongated medical device in the form of an ablation catheter 32. The ablation catheter 32 includes an expandable assembly 34. The expandable assembly 34 includes a plurality of splines 36 each having at least one electrode 38. The plurality of splines 36 form a basket structure that positions the at least one electrode 38 against a tissue surface within a patent. The embodiment illustrated in FIGS. 2A – 2C includes five splines 36, and each of the five splines 36 includes four electrodes 38. Other embodiments include more or less splines 36 and more or less electrodes 38 to facilitate use in various locations of a patient and to achieve a desired purpose. The at least one electrode 38 can take the form of ablation electrodes, mapping electrodes, pacing electrodes, sensing electrodes, or cauterizing electrodes depending on a procedure to be performed.

The outer member 24 of the ablation catheter 32 includes a distal end 40 that is attached to a proximal end of the plurality of splines 36. A distal end of the plurality of splines 36 is attached to a cap 42. In some embodiments, the cap 42 is a separate component. In other embodiments, the cap 42 is formed by molding together the plurality of splines 36. The cap 42 is attached to a distal end of the inner member 28 such that retraction of the inner member causes the plurality of splines 36 to move from a stored configuration as illustrated in FIG. 2A to an expanded configuration as illustrated in FIGS. 2B and 2C.

In FIG. 2A, the stored configuration of the expandable assembly 34 corresponds to the actuator 22 being in a most distal position on the handle 10. In this arrangement, the inner member 28 is also in a most distal position such that the plurality of splines 36 are substantially parallel to a longitudinal axis of the ablation catheter 32.

In FIG. 2B, the expandable assembly 34 of the ablation catheter 32 is in an expanded configuration where the splines 36 form a bow shape. This arrangement corresponds to the actuator 22 being retracted proximally from the most distal position, which in turn retracts the inner member 28.

In FIG. 2C, the expandable assembly 34 of the ablation catheter 32 is in an expanded configuration where the splines 36 form a petal configuration. This arrangement corresponds to the actuator 22 being retracted to a most proximal position on the handle 10, and the inner member 28 it its most proximal position.

The actuator 22 is capable of being moved from a most proximal position to a most distal position, and vice versa, by a user pushing or pulling the actuator. The expandable assembly 34 of the ablation catheter 32 moves between the stored configuration to the petal configuration as the actuator 22 is translated. A user is able to leave the actuator 22 in any location between the most distal positron and the most proximal position in order to expand the splines 36 a desired amount between the stored configuration and the petal configuration.

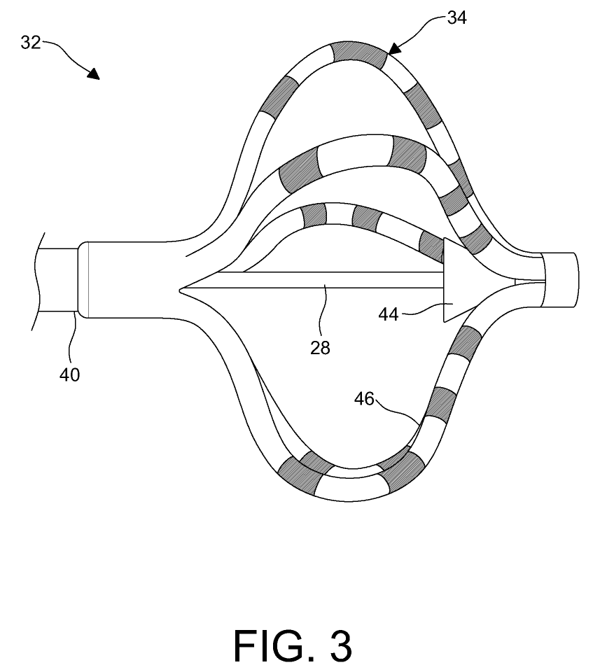

FIG. 3 is a perspective view of a distal portion of an ablation catheter 32 having a bumper 44 positioned on the inner member 28 within the expandable assembly 34 to prevent spline inversion, in accordance with embodiments of the disclosure. The bumper 44 is positioned within expandable assembly 34 in order to prevent the splines 36 from prolapsing or inverting during use. The bumper 44 is configured to abut an inner surface 46 of a distal end portion of the plurality of splines 36 to prevent inversion of the splines 36.

In some embodiments, the bumper 44 is formed of a polymeric material such as silicone or PEBAX. In some embodiments, the bumper 44 is formed a compliant material that can deform or change shape in response to external forces and return to its original state when those forces are removed. Such a material is beneficial when inserting the catheter 32 into a sheath, allowing the splines 36 to press into the bumper 44. In some embodiments, the bumper 44 includes one or more slit extending entirely through the bumper 44 and/or one or more groove that removes a portion of the bumper 44 to enhance collapsibility. The one or more slit or the one or more groove allows the bumper 44 to conform to a space inside of the expandable assembly 34 during insertion of the catheter 32 into a sheath.

In some embodiments, the bumper 44 is formed by molding or extrusion, then placed on the inner member 28 and tacked into position through a reflow or adhesive operation. The bumper 44 is slid longitudinally along the inner member 28 to a desired location, or the bumper 44 can include a slit allowing the bumper 44 to be placed at the desire location. The bumper 44 is tacked into position contacting the inner surface 46 of a distal end portion of the plurality of splines 36. Alternatively, the bumper 44 is formed of a heat shrinkable material and secured at a desired location on the inner member 28 using heat. In other embodiments, the bumper 44 is integral with the inner member 28 and formed by an increased diameter portion of the inner member 28. This can be done by grinding or otherwise shaping the inner member 28.

FIG. 4A is a perspective view of a cylindrical bumper 54 in accordance with an embodiment of the disclosure. The cylindrical bumper 54 includes a bumper proximal end 56, an opposite bumper distal end 58, and an outer profile 60 extending from the bumper distal end 58 to the bumper proximal end 56. In the embodiment of FIG. 4A, the outer profile 60 is consistent. That is, the cylindrical bumper 54 has a consistent diameter between the bumper proximal end 56 and the bumper distal end 58. The cylindrical bumper 54 includes a lumen 62 configured to receive the inner member 28. In some embodiments, the bumper 54 includes a slit 63 allowing the cylindrical bumper 54 to be opened up and placed on the inner member 28 at a desired location. The slit 63 also allows the bumper 54 to flex or conform to a space within the expandable assembly 34 during insertion as discussed above. The cylindrical bumper 54 is installed on the inner member 28 such that a portion of the cylindrical bumper 54 abuts an inner surface 46 of a distal end portion of the plurality of splines 36 to prevent inversion of the splines 36. The splines 36 are prevented from prolapsing or inverting as a result of the cylindrical bumper 54 preventing further movement of the splines across a central axis of the expandable assembly 34.

FIG. 4B is a perspective view of a conical bumper 64 in accordance with an embodiment of the disclosure. The conical bumper 64 includes a bumper proximal end 66, an opposite bumper distal end 68, and an outer profile 70 extending from the bumper distal end 68 to the bumper proximal end 66. The outer profile 70 tapers from a first smaller diameter at the bumper distal end 68 to a second larger diameter at the bumper proximal end 66. The conical bumper 64 includes a lumen 72 configured to receive the inner member 28. In some embodiments, the conical bumper 64 includes a slit 73 allowing the conical bumper 64 to be opened up and placed on the inner member 28 at a desired location. The slit 73 also allows the bumper 64 to flex or conform to a space within the expandable assembly 34 during insertion as discussed above. The conical bumper 64 is installed on the inner member 28 such that a portion of the conical bumper 64 abuts an inner surface 46 of a distal end portion of the plurality of splines 36 to prevent inversion of the splines 36. The splines 36 are prevented from prolapsing or inverting as a result of the conical bumper 64 preventing further movement of the splines across a central axis of the expandable assembly 34.

FIG. 4C is a perspective view of a bumper 74 in accordance with an embodiment of the disclosure. The bumper 74 includes a bumper proximal end 76 and an opposite bumper distal end 78. The bumper 74 includes a first portion 75 having a constant diameter and a second portion 77 having a diameter that tapers towards the bumper distal end 78. The first portion 75 is proximal of the second portion 77. The first portion extends from the proximal end 76 to a shoulder 79. The shoulder 79 has a diameter that is larger than the diameter of the first portion 75. The second portion 77 tapers from the shoulder 79 towards a smaller diameter at the bumper distal end 78. The bumper 74 includes a lumen 82 configured to receive the inner member 28. In some embodiments, the bumper 74 includes a slit 83 allowing the bumper 74 to be opened up and placed on the inner member 28 at a desired location. The slit 83 also allows the bumper 74 to flex or conform to a space within the expandable assembly 34 during insertion as discussed above. The bumper 74 is installed on the inner member 28 such that a portion of the bumper 74 abuts an inner surface 46 of a distal end portion of the plurality of splines 36 to prevent inversion of the splines 36. The splines 36 are prevented from prolapsing or inverting as a result of the bumper 74 preventing further movement of the splines across a central axis of the expandable assembly 34.

FIG. 4D is a perspective view of a bumper in accordance with an embodiment of the disclosure. The bumper 84 includes a bumper proximal end 86 and an opposite bumper distal end 88. The bumper 84 includes a first portion 85 having a first constant diameter, a second portion 87 having a second constant diameter, and a third portion 89 having a third constant diameter. The second constant diameter is larger than both the first constant diameter and the third constant diameter. In some embodiments, the first and third constant diameter are the same. In some embodiments, the first constant diameter is larger than the third constant diameter. In some embodiments, the first constant diameter is smaller than the third constant diameter. The first portion 85 is distal of the second portion 87, and the second portion 87 is distal of the third portion 89. The bumper 84 includes a lumen 92 configured to receive the inner member 28. In some embodiments, the bumper 84 includes a slit 93 allowing the bumper 84 to be opened up and placed on the inner member 28 at a desired location. The slit 93 also allows the bumper 84 to flex or conform to a space within the expandable assembly 34 during insertion as discussed above. The bumper 84 is installed on the inner member 28 such that a portion of the bumper 84 abuts an inner surface 46 of a distal end portion of the plurality of splines 36 to prevent inversion of the splines 36. The splines 36 are prevented from prolapsing or inverting as a result of the bumper 84 preventing further movement of the splines across a central axis of the expandable assembly 34.

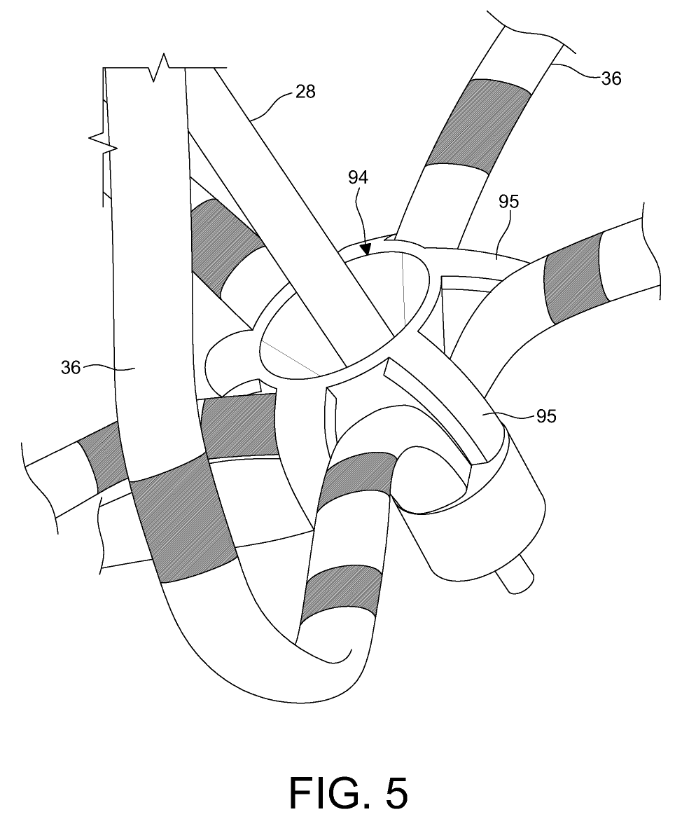

FIG. 5 is a perspective view of a distal portion of an ablation catheter having a bumper 94 with a plurality of fins 95, in accordance with embodiments of the disclosure. The plurality of fins 95 prevent bunching of the splines 36 when the expandable assembly 34 is manipulated around a curve or pressed against anatomy.

Referring to FIGS. 6A and 6B, the bumper 94 includes a bumper proximal end 96 and an opposite bumper distal end 98. The bumper 94 tapers from a larger diameter at the bumper proximal end 96 to a smaller diameter at the bumper distal end 98. The plurality of fins 95 are spaced apart along the outer profile of the bumper 94. As illustrated in FIG. 6A, the plurality of fins 95 are evenly spaced apart around the surface of the bumper 94. Each of the plurality of fins 95 are separated by a groove 97 configured to receive one of the splines 36. When assembled, each groove 97 holds one of the splines 36, and each of the splines 36 are separated by one of the plurality of fins 95. The bumper 94 includes a lumen 102 configured to receive the inner member 28. In some embodiments, the bumper 94 includes a slit (not shown) allowing the bumper 94 to be opened up and placed on the inner member 28 at a desired location. While bumper 94 is illustrated as having five grooves 97 and five fins 95, it is understood that a bumper can include more or less fins and grooves to accommodate an expandable assembly having more or less splines.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A medical device, the medical device comprising:
an outer member having a proximal end and an opposite distal end;
an inner member movable within the outer member;
an expandable assembly extending distally from the distal end of the outer member, the expandable assembly including a distally located central hub portion affixed to the inner member and a plurality of splines, each of the plurality of splines including a distal end portion extending from the central hub portion, and a proximal end portion attached to the distal end of the outer member;
at least one electrode located on each of the plurality of splines; and
a bumper positioned on the inner member within the expandable assembly, the bumper configured to abut an inner surface of the distal end portion of the plurality of splines to prevent inversion of the plurality of splines.

2. The medical device of claim 1, wherein the at least one electrode is an ablation electrode, sensing electrode, mapping electrode, pacing electrode, or cauterizing electrode.

3. The medical device of claim 1, wherein the bumper includes a bumper proximal end, an opposite bumper distal end, and an outer profile extending from the bumper distal end to the bumper proximal end.

4. The medical device of claim 3, wherein the outer profile tapers from a smaller diameter at the bumper distal end to a larger diameter at the bumper proximal end.

5. The medical device of claim 1, wherein the bumper includes a plurality of fins, the plurality of fins being spaced apart along the outer profile.

6. The medical device of claim 5, wherein the plurality of fins are configured to be received between adjacent ones of the plurality of splines.

7. The medical device of claim 1, wherein the bumper is a polymeric material.

8. The medical device of claim 7, wherein the polymeric material is silicone or PEBAX.

9. The medical device of claim 1, wherein the bumper is cylindrical or conical.

10. The medical device of claim 1, wherein the bumper includes a lumen configured to receive the inner member.

11. The medical device of claim 1, wherein the bumper is formed of a compliant material.

12. The medical device of claim 1, wherein the bumper includes a first portion having a first diameter, and a second portion having a second diameter that is larger than the first diameter.

13. The medical device of claim 12, wherein the first portion is distal to the second portion.

14. The medical device of claim 1, wherein the inner member has a portion of increased diameter forming the bumper.

15. A The medical device, the medical device comprising:
an outer member having a proximal end and an opposite distal end;
an inner member movable within the outer member;
an expandable assembly extending distally from the distal end of the outer member, the expandable assembly including a distally located central hub portion affixed to the inner member and a plurality of splines, each of the plurality of splines including a distal end portion extending from the central hub portion, and a proximal end portion attached to the distal end of the outer member; and
a cylindrical or conical bumper positioned on the inner member within the expandable assembly, the bumper configured to abut an inner surface of the distal end portion of the plurality of splines to prevent inversion of the plurality of splines.

16. The medical device of claim 15, further comprising at least one electrode located on each of the plurality of splines.

17. The medical device of claim 16, wherein the at least one electrode is an ablation electrode, sensing electrode, mapping electrode, pacing electrode, or cauterizing electrode.

18. A medical device, the medical device comprising:
an outer member having a proximal end and an opposite distal end;
an inner member movable within the outer member;
an expandable assembly extending distally from the distal end of the outer member, the expandable assembly including a distally located central hub portion affixed to the inner member and a plurality of splines, each of the plurality of splines including a distal end portion extending from the central hub portion, and a proximal end portion attached to the distal end of the outer member; and
a bumper positioned on the inner member within the expandable assembly, the bumper configured to abut an inner surface of the distal end portion of the plurality of splines to prevent inversion of the plurality of splines, the bumper including a first portion having a first diameter, and a second portion having a second diameter that is larger than the first diameter.

19. The medical device of claim 18, wherein the first portion is proximal of the second portion.

20. The medical device of claim 18, wherein the first portion is distal of the second portion.
